(19) United States
(12) Patent Application Publication
(10) Pub. No.: US 2021/0012888 A1
(43) Pub. Date: Jan. 14, 2021

(54) COMMAND SYSTEM USING DATA CAPTURE AND ALERTS

(71) Applicant: Tagnos, Inc., Irvine, CA (US)

(72) Inventor: Jagadesh Appla Padala, Irving, TX (US)

(21) Appl. No.: 16/510,504

(22) Filed: Jul. 12, 2019

(51) Int. Cl. G16H 40/20; G16H 40/63; G16H 10/60

(52) U.S. Cl. CPC G16H 40/20; G16H 10/60; G16H 40/63

(57) ABSTRACT

Systems and methods are disclosed for improving healthcare services in a healthcare facility, including multiple overlapping operations requiring or requesting multiple overlapping healthcare resources for multiple patients, accounting for and adjusting to real time delays and changes to the workflow for each procedure. Live datasets related to one or more patients related to a procedure scheduled for the patient is accessed, including patient data, healthcare personnel, healthcare resources, healthcare infrastructures, and tasks related to advancing a patient through the workflow for the procedure. Healthcare personnel associated with a task in the workflow are notified of a deficiency related to the task, and the healthcare personnel corrects the deficiency. The live data set is updated with corrected status of the deficiency. Elements for the live dataset for one or more patients is displayed to healthcare personnel, and is updated as changes to the workflow or data elements occur.

### FIELD OF THE INVENTION

The field of the invention is healthcare facilities and activities.

### BACKGROUND

Managing, orchestrating, or optimizing work flows that involve multiple prerequisites, tasks, participants, or resources with disparate lead and lag times is a substantial problem for large facilities or enterprises, and as such an area of great interest and development. However, designing, improving, or implementing such workflows is incredibly complicated and often yields suboptimal or unacceptable results due to cost-prohibitive or insurmountable challenges. Such complications are compounded in industries where multiple, concurrent operations are performed that require overlapping resources with shared upstream or downstream tasks, all occurring within a confined space (e.g., manufacturing facilities, construction sites, healthcare facilities, etc). The burden of designing, improving, or implementing workflows grows exponentially in healthcare facilities, where modifying workflows can have life or death affects on patients and where the results of individual activities (e.g., surgery, treatment outcome, etc) are largely unknown or unpredictable, and can be interrupted by unanticipated emergencies.

Efforts have been made to address the dilemmas faced in managing, orchestrating, or optimizing complex workflows in healthcare facilities. For example, U.S. Pat. No. 7,492,266 to Bhavani et al (the '266 patent), and likewise US Publication No. 2009/0051546 to Bhavani (the '546 application), teaches improving workflows using VoIP and RFID tags to improve communication between doctors, nurses, and healthcare resources and providing updated information regarding resources. However, while simply improving communication and updating information may improve performance of workflows, they offer little or no aid in the design of workflows, or the improvement and optimization of those workflows. Further, the teachings of the '266 patent require doctors or nurses to actively initiate the system and communicate, therefore lacking the benefits of passive, background, or automated management systems.

These and all other publications referenced herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Commercial efforts have been made to streamline and refine the organization and display of data related to the multiple steps in a healthcare procedure for a patient. For example, the OR Cockpit product available from Skytron® attempts to organize critical data related to a patient advancing through the workflow for a procedure in a readily ascertainable display. See web address skytron.us/products/pages/visual_management_solutions.html, viewed Jul. 10, 2019. However, the Skytron system does not appear adapted to notify healthcare workers in the field or throughout the healthcare environment, or allow automated or manual push notifications regarding various elements related to the workflow of a procedure to be received and updated, or display data related to multiple patients, or recommend changes to the workflow based on real time status for each patient.

Thus, there remains a need for methods and systems to collect, organize, and present real time data related to the workflow for procedures for one or more patients in a health care facility.

### SUMMARY OF THE INVENTION

The inventive subject matter contemplates kits, devices, systems, and methods for improving healthcare within a healthcare facility, in particular relating to a procedure (e.g., surgical operation, etc) on a patient. A live dataset is accessed, which includes up-to-date data (e.g., real time, contemporaneous, accurate as of most recent change, etc) regarding (i) a status of the patient, (ii) a status of a plurality of healthcare assets related to the procedure, (iii) a status of a plurality of infrastructure elements of the healthcare facility related to the procedure, and (iv) a status of a plurality of tasks required to advance the patient through a workflow of the procedure. Contemplated live data sets include those disclosed in copending U.S. application Ser. No. 16/190,681 and copending US Publication No. US 2018/0204640 by the present inventors, and such methods and systems disclosed therein.

A healthcare worker (e.g., administrative staff, doctor, nurse, custodian, technician, mechanic, etc) associated with a first task from the plurality of tasks is notified of a deficiency in the live dataset regarding at least one of (i) a status of the patient related to the first task, (ii) a status of a first healthcare asset from the plurality of healthcare assets related to the first task, (iii) a status of a first infrastructure element from the plurality of infrastructure elements related to the first task, or (iv) a status of a second task from the plurality of tasks that is related to the first task. While the healthcare worker may be notified at a central display in the healthcare facility, it is also contemplated that the healthcare worker receive notification over a PA system or on a personal device, for example via an app on the worker's smart phone. The healthcare worker then corrects the deficiency, which is preferably either automatically updated in the live dataset or manually updated by the healthcare worker, for example via an app on the patient's smart phone.

Systems for presenting live status of a first procedure for a first patient to personnel in a healthcare facility a further contemplated. A processor accesses a live dataset including (i) a status of the first patient, (ii) a status of a plurality of healthcare assets related to the first procedure, (iii) a status of a plurality of infrastructure elements of the healthcare facility related to the first procedure, and (iv) a status of a plurality of tasks required to advance the first patient through a workflow of the first procedure. A visual display that is informationally coupled with the processor displays elements from the live dataset, including for example at least part of (i) the status of the first patient, (ii) the status of the plurality of healthcare assets related to the first procedure, (iii) the status of the plurality of infrastructure elements of the healthcare facility related to the first procedure, and (iv) the status of the plurality of tasks required to advance the first patient through the workflow of the first procedure, though it is contemplated that the display can include all such information regarding the first patient and the first procedure, a summary of all such information, or a curated or user selected collection of such information.

A communication module that is informationally coupled with the processor then notifies a healthcare worker associated with a first task from the plurality of tasks of a deficiency related to that task that has been identified in the live dataset (for example by the processor). The healthcare worker then corrects the deficiency, and the visual display updates the displayed elements from the live dataset to include the corrected deficiency, which is preferably automatically updated in the live dataset or manually updated by the healthcare worker.

Methods for advancing a patient through a workflow of a procedure in a healthcare facility are also contemplated. A live dataset is accessed, which includes data regarding (i) a status of the patient, (ii) a status of a plurality of healthcare assets related to the procedure, (iii) a status of a plurality of infrastructure elements of the healthcare facility related to the procedure, and (iv) a status of a plurality of tasks required to advance the patient through the workflow of the procedure. Simultaneously (or nearly simultaneous, or substantially simultaneous) a first healthcare worker associated with a first task in a first step of the workflow is notified of a deficiency related to the first task, and a second healthcare worker of a second task in a subsequent second step in the workflow is notified of a deficiency related to the second task. The first and second health workers correct the deficiencies in the first and second tasks, respectively, such that the patient is cleared to progress from the first step of the workflow, through the second step of the workflow, to a subsequent third step in the workflow, which is preferably related to additional healthcare resources and further (or the same) healthcare workers.

### BRIEF DESCRIPTION OF THE DRAWING

FIGS. 1A, 1B, and 1C depict panels of a user display of the inventive subject matter.

FIG. 2 depicts an additional user display of the inventive subject matter.

FIG. 3 depicts an additional user display of the inventive subject matter.

### DETAILED DESCRIPTION

The inventive subject matter contemplates kits, devices, systems, and methods for improving healthcare within a healthcare facility, in particular relating to a procedure (e.g., surgical operation, etc) on a patient. A live dataset is accessed, which includes up-to-date data (e.g., real time, contemporaneous, accurate as of most recent change, etc) regarding (i) a status of the patient, (ii) a status of a plurality of healthcare assets related to the procedure, (iii) a status of a plurality of infrastructure elements of the healthcare facility related to the procedure, and (iv) a status of a plurality of tasks required to advance the patient through a workflow of the procedure. Contemplated live data sets include those disclosed in copending U.S. application Ser. No. 16/190,681 and copending US Publication No. US 2018/0204640 by the present inventors, and such methods and systems disclosed therein. A healthcare worker (e.g., administrative staff, doctor, nurse, custodian, technician, mechanic, ect) associated with a first task from the plurality of tasks is notified of a deficiency in the live dataset regarding at least one of (i) a status of the patient related to the first task, (ii) a status of a first healthcare asset from the plurality of healthcare assets related to the first task, (iii) a status of a first infrastructure element from the plurality of infrastructure elements related to the first task, or (iv) a status of a second task from the plurality of tasks that is related to the first task. The healthcare worker then corrects the deficiency, which is preferably either automatically updated in the live dataset or manually updated by the healthcare worker.

The status of the patient recorded in the live dataset is generally at least one of an administrative detail (e.g., patient check-in, patient registration, patient MRN, patient insurance coverage, etc) a pre-procedure detail (e.g., patient pre-procedure condition, patient pre-procedure location, completion of pre-procedure instructions to patient (e.g., fasting), etc), or a post-procedure detail (e.g., patient post-procedure condition, patient post-procedure location, post-procedure instruction to patient (e.g., bed rest, follow up labs, prescription, etc), or patient discharge, etc).

The status of healthcare assets recorded in the live dataset is generally relates to at least (i) a healthcare worker (e.g., admin, nurse, doctor, technician, custodian, etc) related to a pre-procedure, procedure, or post-procedure task, (ii) a report (e.g., lab, radiology, etc) related to one of a pre-procedure, procedure, or post-procedure task, or (iii) a hardware (e.g., cart for specific procedure (vascular graft cart), imaging unit for procedure (e.g., Doppler ultrasound unit), cart specialized for healthcare worker (e.g., specific to doctor X), etc) related to one of a pre-procedure, procedure, or post-procedure task. The healthcare assets can be downstream from the current position of the patient in a workflow for the procedure (e.g., preparing for next steps in procedure workflow), can be assets needed or used in the current position of the patient in the workflow (e.g., status of currently used assets, account for or count assets), or can be assets that were previously used (e.g., attend to sanitation, remediation, or cleanup of used or soiled assets for next use).

The status of infrastructure elements recorded in the live dataset relates to at least one of HVAC, temperature for procedure room (e.g., operating room), humidity of procedure room, entries into procedure room, number of door openings (times door opened) in procedure room, or status of a post-procedure room (e.g., post-op bed, inpatient care, discharge room, etc).

The status of tasks required to advance the patient through the workflow of the procedure include healthcare facility steps (e.g., clinical, administrative, custodial, technical, etc) related to (i) pre-procedure, procedure, or post-procedure, (ii) safety checks related to pre-procedure, procedure, or post-procedure (e.g., surgery checks, counting work pieces, AB protocol, lab, Xray, EKG, Anesthesiologist check, (iii) environmental checks (e.g., room temperature, room humidity, number of times door opened, etc), or (iv) sanitation steps related to pre-procedure, procedure, or post-procedure (e.g., clean pre-procedure, procedure, or post-procedure room, clean assets used in procedure, pre-procedure, or post-procedure, etc).

In some embodiments, the live dataset includes information regarding a second patient for a second procedure, similar to the information recorded for the first patient or first procedure. The second procedure can occur after or before the first procedure for the first patient, and in some embodiments the second procedure requires healthcare facility resources (e.g., rooms, workers, equipment, beds, lab time, admin time, etc) that at least partially overlaps with the procedure. Viewed from another perspective, the inventive subject matter contemplates methods systems were multiple healthcare resources are required, requested, or allocated for multiple procedures between multiple patients, and workflows are designed, tracked, and corrected to accommodate such multiplicity of operations.

Preferably, the live dataset is updated contemporaneously (or nearly) by push updates from each healthcare assets (e.g., equipment, worker, lab, etc) or infrastructure element of the facility (e.g., HVAC, environmental elements, rooms, etc). While it is contemplated that the healthcare worker directly corrects the identified deficiency (e.g., performs required safety check, sanitation task, procedure task, etc), in some embodiments the deficiency is indirectly corrected by the healthcare worker, for example by instructing, notifying, or alerting another worker to the deficiency (e.g., peer worker, higher skilled worker, lowered skilled worker, worker with different skill set or job responsibilities, etc), or attending to the deficiency through automated systems or peripheral controls (e.g., adjust temperature or humidity via controls or automated system), etc.

In some embodiments, the healthcare worker changes a part of the workflow of the procedure based on the deficiency, for example rescheduling the procedure, scheduling additional labs, scheduling additional or different healthcare assets for the pre-procedure, procedure, or post-procedure (e.g., complications arise during procedure requiring additional procedure or additional post-procedure steps, staff or doctor availability changes, etc). In some embodiments, changing the part of the workflow is recommended by an automated management system (e.g., Tagnos system, see copending U.S. application Ser. No. 16/190,681 and copending US Publication No. US 2018/0204640 by the present inventors) of the healthcare facility and consists of rescheduling the procedure or changing elements of the procedure or assets required for the procedure.

The second task can be either upstream or downstream of the first task in the workflow for the procedure. Preferably, the second task is one of a pre-procedure task or a procedure task, while the first task is one of a procedure task or a post-procedure task, but the opposite or other combinations are contemplated.

Some embodiments further include visually displaying elements (e.g., all, some, a preset group, a curated group, a user selected group, a summary, etc) of the live dataset including at least part of (i) the status of the patient, (ii) the status of the healthcare assets related to the procedure, (iii) the status of the infrastructure elements of the healthcare facility related to the procedure, and (iv) the status of tasks required to advance the patient through the procedure.

A safety score is further calculated in some embodiments, for example a safety score for the procedure for the patient based on all or part of the live dataset. Preferably, an updated safety score for the procedure for the patient is calculated each time the live dataset is updated, for updated with correcting the deficiency.

Systems for presenting live status of a first procedure for a first patient to personnel in a healthcare facility a further contemplated. A processor accesses a live dataset including (i) a status of the first patient, (ii) a status of a plurality of healthcare assets related to the first procedure, (iii) a status of a plurality of infrastructure elements of the healthcare facility related to the first procedure, and (iv) a status of a plurality of tasks required to advance the first patient through a workflow of the first procedure. A visual display that is informationally coupled with the processor displays elements from the live dataset, including for example at least part of (i) the status of the first patient, (ii) the status of the plurality of healthcare assets related to the first procedure, (iii) the status of the plurality of infrastructure elements of the healthcare facility related to the first procedure, and (iv) the status of the plurality of tasks required to advance the first patient through the workflow of the first procedure, though it is contemplated that the display can include all such information regarding the first patient and the first procedure, a summary of all such information, or a curated or user selected collection of such information. A communication module that is informationally coupled with the processor then notifies a healthcare worker associated with a first task from the plurality of tasks of a deficiency related to that task that has been identified in the live dataset (for example by the processor). The healthcare worker then corrects the deficiency, and the visual display updates the displayed elements from the live dataset to include the corrected deficiency, which is preferably automatically updated in the live dataset or manually updated by the healthcare worker.

In some embodiments, the live status of a second procedure for a second patient is presented to personnel, such that the live dataset includes information similar to the first patient and the first procedure and the live status of the second patient is derived from the live dataset. In some embodiments, the first procedure for the first patient is scheduled to occur before the second procedure for the second patient, and the first procedure and the second procedure require at least one common healthcare facility asset.

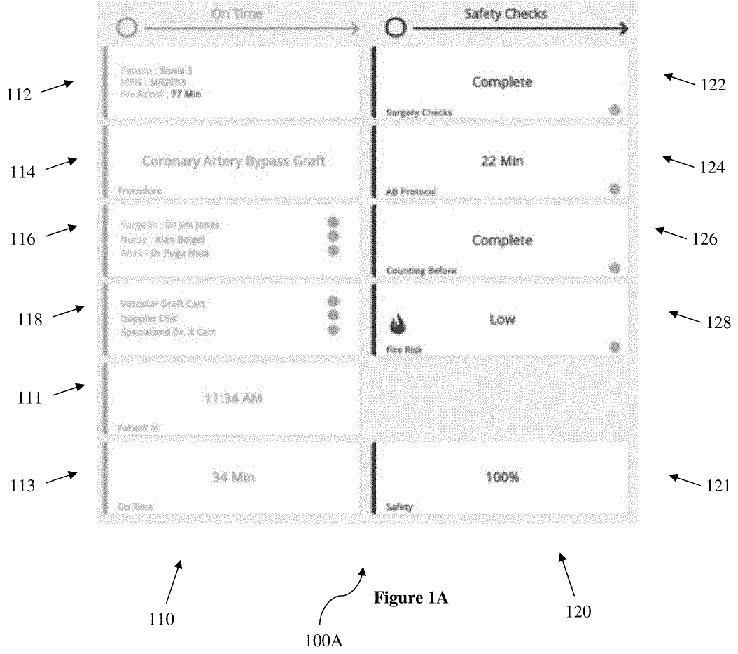

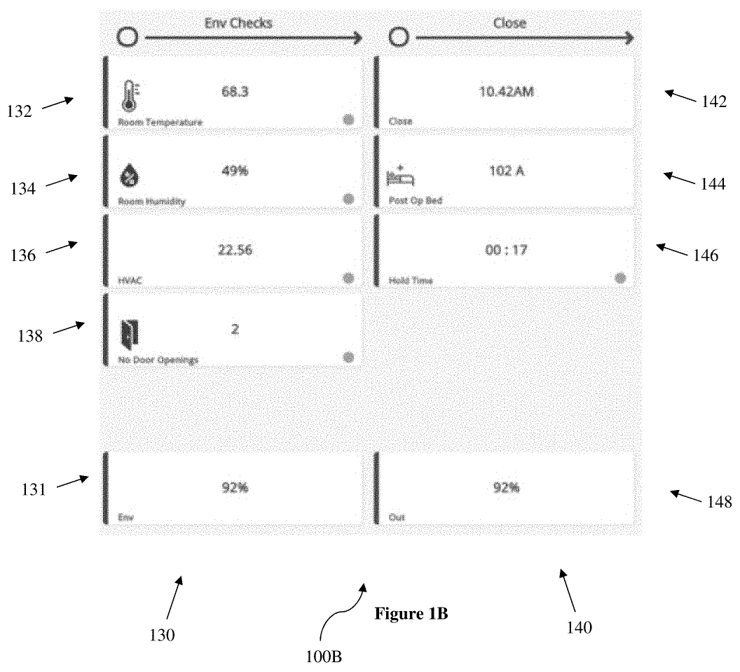

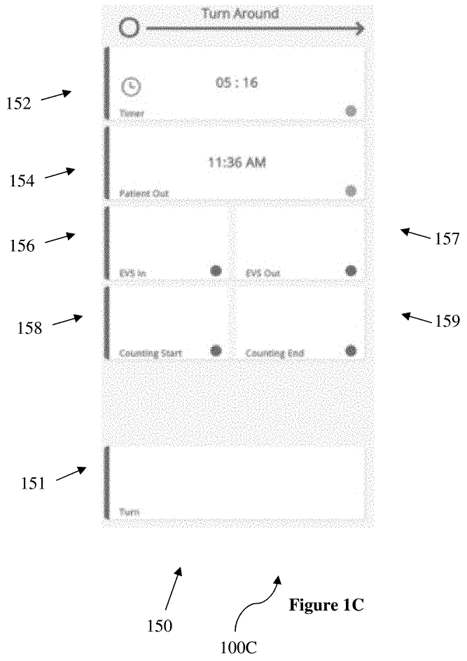

FIGS. 1A, 1B, and 1C depict panels of a user display of the inventive subject matter, which are preferably displayed together on a single display, or an array of adjacent displays, from left to right, FIGS. 1A, 1B, and 1C. FIG. 1A depicts panel 100A, which includes procedure status at column 110 and safety status at column 120. Column 110 lists patient information in box 112, including the name of the patient (Sonia S), the medical record number for the patient (MR2058), and the predicted time to complete the procedure, including pre-procedure and post-procedure events (77 minutes). It is contemplated that the predicted time is estimated by a healthcare facility management system, for example those described in copending U.S. application Ser. No. 16/190,681 to the present inventors. The time for completing the procedure is colored red to indicate to the user that the time is outside of the estimated or budgeted time for this particular procedure. When the time is within or below the estimated or budgeted time, the number is green, and when it is within a reasonable margin from the budgeted time (e.g., within 5%, 10%, 15%, 20%), the number is yellow.

Box 114 indicates the type of procedure that will performed on the patient, here a coronary artery bypass graft. Box 116 indicates the healthcare facility personnel that are assigned to this procedure for this patient, here including surgeon, nurse, and anesthesiologist, as well as the status for each person, here green for each. In the case where a person assigned to the procedure is not prepared or ready for the procedure, the indicated is another color (example yellow, red, or black). While surgeon, nurse, and anesthesiologist are identified for this procedure, it should be appreciated that any healthcare personnel required or requested for each specific procedure can be identified in box 116.

Box 118 describes the healthcare equipment required or requested for the procedure and the status of each piece of equipment. Here, vascular graft cart, Doppler unit, and specialized doctor X cart are identified, with each indicated as green to represent the equipment is ready or in place for the procedure (e.g., in the operating room). Again, where a piece of equipment is not ready, the indicator is another color, for example yellow, red, or black. Also, as many or as few pieces of equipment that are required or requested for the procedure can be identified in box 118.

Box 111 depicts the patient in time (11:34 am), which can be used to determine the overall efficiency of the workflow for the procedure. Box 113 depicts what the budgeted, on time duration of the procedure is, here 34 minutes. As can be seen by comparing the predicted time from box 112 and the budgeted time in box 113, the predicted time to complete the procedure is more than double the budgeted time.

Column 120 depicts the safety status for the procedure. Box 122 indicates that surgery checks have been completed, as indicated by the color green. Box 124 indicates the AB protocol has been completed, indicating the time taken to perform the protocol (22 minutes) and a green indicator that the time was within budgeted parameters. Box 126 indicates the counting of work pieces to be used in the operating room (e.g., sponges, scalpel, clamps, etc) have been completed before the procedure, as indicated by the green light. Box 128 indicates that the fire risk during the procedure is low, which is within the desired parameters as indicated by the green light. Box 121 indicates a safety calculation based on the safety checks of column 120. As each of the safety parameters has been completed or is otherwise satisfactory, the safety calculation is at 100%. Where one or more of the safety parameters has not been completed or is otherwise less than satisfactory, the safety calculation will be below 100%. In some embodiments, the procedure will not be permitted to proceed if critical safety parameters are not satisfactory or if the safety score in box 121 is too low (e.g., below 90%, 80%, 70%, or 60%). In general, a satisfactory condition for elements in FIGS. 1A, 1B, and 1C is shown by a green indicator, while less satisfactory or unsatisfactory conditions are indicated by another color, for example yellow, red, gray, or black.

FIG. 1B depicts panel 100B of a user display, which includes environmental status in column 130 and close status in column 140. Box 132 depicts the room temperature for the procedure room (e.g., operating room), which is at 68.3° C., which is within the desired parameter as shown by the green indicator. Box 134 shows the room humidity, which is at 49%, outside of the desired humidity range as indicated by the yellow indicator. It should be appreciated that the temperature and humidity of the procedure room have a direct impact on the chance of infection following the procedure. Preferably, the procedure room will be below 20%, more preferably below 10%, and more preferably below 5% humidity to prevent infection.

Box 136 indicates the status of the HVAC system, which is operating at 22.56, within the desired parameter as indicated by the green indicator. Box 138 shows the number of times the door or doors to the procedure room (e.g., operating room) have been opened in the past 5, 10, 15, 20, 25, 30, 45, or 60 minutes, or during the duration of the procedure. Here, two openings is outside of the desired parameter, as shown by the yellow indicator. In general, it is desirable to keep door openings to a minimum before and during a procedure. Box 131 shows a score of the environmental factors related to the procedure, here 92%. It should be apparent that the unsatisfactory humidity condition in box 134 and unsatisfactory door opening condition in box 138 contributed to an environmental score of less than 100%. In some embodiments, a low environmental score (e.g., below 90%, 80%, 70%, 60%, or 50%) will prevent the patient from progressing through the workflow for the procedure.

Column 140 depicts the close status for the procedure that occurred before the procedure scheduled for the patient in column 110 of FIG. 1A. Box 142 shows the time the prior patient's procedure closed (10:42 am). Box 144 shows the post procedure resource the prior patient is using, here post op bed 102A. Box 146 shows the amount of time the prior patient has been held, here 17 minutes, which is outside of the satisfactory parameter. In some embodiments, a patient should be held for less than 17 minutes, for example where the procedure is minimally invasive and does not require post-procedure observation. However in some embodiments the patient should be held for more than 17 minutes, for example where the procedure was extensive or traumatic, requiring an extended period of post-procedure observation. Box 148 indicates a score for the closing procedure of the prior patient, here 92%. Here, an unsatisfactory hold time contributed to the closing score being less than 100%.

FIG. 1C depicts panel 100C of a user display, which includes the turnaround time for the patient procedure proceeding the patient procedure identified in column 110. Box 152 includes the time for completion of pre-procedure, procedure, and post-procedure processes for the previous patient, 5 hours and 16 minutes, which is within the desired parameters as shown by the green indicator. Box 154 indicates the patient out time for the prior patient, 11:36 am, which is within the desired parameter as shown by the green indicator. Boxes 156 and 157 show that EVS activities completed the post-procedure events (e.g., cleaning procedure room), as shown by the indicator for EVS in and EVS out as green (e.g., complete). Boxes 158 and 159 show the counting start and counting end for items used during the procedure have been completed, and the outcome was within the desired parameters (e.g., all items accounted for), as shown by the green indicators. Box 151 indicates that turnaround of the prior patient has been complete, as shown by the green border of the box. In preferred embodiments, the status of elements in FIGS. 1A, 1B, and 1C is automatically tracked and recorded, for example per the teachings of copending U.S. application Ser. No. 16/190,681 and copending US Publication No. US 2018/0204640 by the present inventors, or manually entered by healthcare facility personnel, or a combination of both.

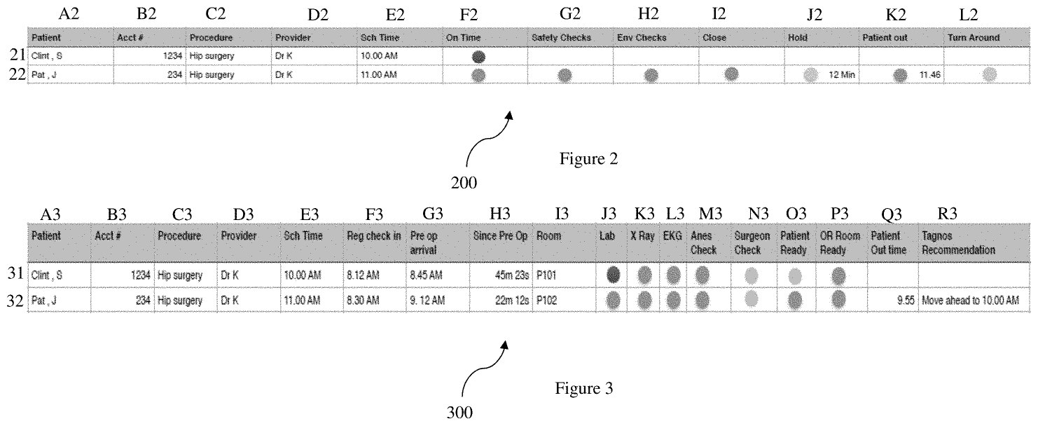

FIG. 2 depicts table 200 displaying patient data related to a procedure, with column A2 displaying the patient's name, column B2 displaying the patient's account number, column C2 displaying the procedure for the patient, column D2 displaying the provider for the procedure, column E2 displaying the scheduled time for the procedure, column F2 showing the on-time status for the patient, column G2 showing the safety check status for the procedure, column H2 showing the environmental check status for the procedure, column I2 showing the close status for the procedure, column J2 showing the post-procedure hold status for the patient, and column K2 showing the patient out status for the patient. Row 21 includes data for patient Clint, S, while row 22 shows data for Pat, J. As depicted, the green indicators in columns F2, G2, H2, and I2 show that patient Pat, J is on time, all safety checks are complete, all environmental checks are complete, close is complete, and the patient has been checked out. Column J2 indicates that the hold period for Pat, J was unsatisfactory at 12 minutes. Column F2 for patient Clint, S shows that Clint, S was not on time, and the empty indicators for columns G2, H2, I2, J2, and K2 indicate that the procedure has not progressed for patient Clint, S.

FIG. 3 depicts table 300 displaying pre-procedure data for patients in row 31 and 32, Clint, S and Pat, J respectively. Column A3 has the patient's name, column B3 has the account number, column C3 has the procedure, column D3 has the provider, column E3 has the scheduled time for each procedure, column F3 has the registered check in time for each patient, column G3 has the arrival time for each patient into pre-procedure, column H3 has the time spent waiting for each patient since entering pre-procedure, column I3 has the room that each patient is assigned to for pre-procedure, column J3 has the status for pre-procedure labs, column K3 has the status for pre-procedure Xrays, column L3 has the status for pre-procedure EKG, column M3 shows the status for anesthesiologist check, column N3 has the status for surgeon check, column O3 has the status for whether patient is ready for the procedure, including all pre-procedure checks, column P3 indicates the status of the operating room for each patient's procedure, column Q3 depicts the expected time for the patient to leave pre-procedure phase, and column R3 depicts that recommendation generated by the healthcare facility management system Tagnos (see copending U.S. application Ser. No. 16/190,681 and copending US Publication No. US 2018/0204640 by the present inventors). Red indicators (column J3, row 31) show the event is incomplete or unsatisfied, green indicators show the event is complete or satisfied, and grey indicates the event is pending or incomplete.

Descriptions throughout this document include information that may be useful in understanding the present invention. It is not an admission that any of the information provided herein is prior art or relevant to the presently claimed invention, or that any publication specifically or implicitly referenced is prior art.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

Unless the context dictates the contrary, all ranges set forth herein should be interpreted as being inclusive of their endpoints, and open-ended ranges should be interpreted to include commercially practical values. Similarly, all lists of values should be considered as inclusive of intermediate values unless the context indicates the contrary. The recitation of ranges of values herein is merely intended to serve as a shorthand method of referring individually to each separate value falling within the range. Unless otherwise indicated herein, each individual value is incorporated into the specification as if it were individually recited herein.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Various objects, features, aspects and advantages of the inventive subject matter will become more apparent from the detailed description of preferred embodiments, along with the accompanying drawing figures in which like numerals represent like components.

It should be noted that any language directed to a computer should be read to include any suitable combination of computing devices, including servers, interfaces, systems, databases, agents, peers, engines, controllers, or other types of computing devices operating individually or collectively. One should appreciate the computing devices comprise a processor configured to execute software instructions stored on a tangible, non-transitory computer readable storage medium (e.g., hard drive, solid state drive, RAM, flash, ROM, etc.). The software instructions preferably configure the computing device to provide the roles, responsibilities, or other functionality as discussed below with respect to the disclosed apparatus. In especially preferred embodiments, the various servers, systems, databases, or interfaces exchange data using standardized protocols or algorithms, possibly based on HTTP, HTTPS, AES, public-private key exchanges, web service APIs, known financial transaction protocols, or other electronic information exchanging methods. Data exchanges preferably are conducted over a packet-switched network, the Internet, LAN, WAN, VPN, or other type of packet switched network.

The discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A method of improving healthcare within a healthcare facility related to a procedure on a patient, comprising:
accessing a live dataset including (i) a status of the patient, (ii) a status of a plurality of healthcare assets related to the procedure, (iii) a status of a plurality of infrastructure elements of the healthcare facility related to the procedure, and (iv) a status of a plurality of tasks required to advance the patient through a workflow of the procedure;
notifying a healthcare worker associated with a first task from the plurality of tasks of a deficiency in at least one of (i) a status of the patient related to the first task, (ii) a status of a first healthcare asset from the plurality of healthcare assets related to the first task, (iii) a status of a first infrastructure element from the plurality of infrastructure elements related to the first task, or (iv) a status of a second task from the plurality of tasks that is related to the first task; and
correcting, by the healthcare worker, the deficiency.

2. The method of claim 1, wherein the status of the patient consists of at least one of patient check-in, patient registration, patient pre-procedure condition, patient pre-procedure location, patient post-procedure condition, patient post-procedure location, or patient discharge.

3. The method of claim 1, wherein the plurality of healthcare assets consists of at least one of (i) a healthcare worker related to one of a pre-procedure, procedure, or post-procedure task, (ii) a report related to one of a pre-procedure, procedure, or post-procedure task, or (iii) a hardware related to one of a pre-procedure, procedure, or post-procedure task.

4. The method of claim 3, wherein the plurality of healthcare assets are downstream from a current status of the patient in a workflow of the procedure.

5. The method of claim 1, wherein the plurality of infrastructure elements consists of at least one of HVAC, procedure room temperature, procedure room humidity, entries into procedure room, or post-procedure room.

6. The method of claim 1, wherein the plurality of tasks required to advance the patient through the workflow of the procedure consists of healthcare facility steps related to (i) pre-procedure, procedure, or post-procedure, (ii) safety checks related to pre-procedure, procedure, or post-procedure, or (iii) sanitation steps related to pre-procedure, procedure, or post-procedure.

7. The method of claim 1, further comprising accessing a live dataset regarding a second patient for a second procedure, wherein the second procedure occurs after the procedure for the patient, and the second procedure requires healthcare facility resources that at least partially overlap with the procedure.

7. The method of claim 1, wherein the live dataset includes updates pushed from the plurality of healthcare assets or the plurality of infrastructure elements.

8. The method of claim 1, wherein the step of correcting the deficiency is performed indirectly by the healthcare worker.

9. The method of claim 1, further comprising the healthcare worker changes a part of the workflow of the procedure based on the deficiency.

10. The method of claim 9, wherein changing the part of the workflow is recommended by an automated management system of the healthcare facility and consists of rescheduling the procedure.

11. The method of claim 1, wherein the second task is upstream of the first task in the workflow for the procedure.

12. The method of claim 11, wherein the second task is one of a pre-procedure task or a procedure task, and the first task is one of a procedure task or a post-procedure task.

13. The method of claim 1, further comprising visually displaying a plurality of elements of the live dataset including at least part of (i) the status of the patient, (ii) the status of the plurality of healthcare assets related to the procedure, (iii) the status of the plurality of infrastructure elements of the healthcare facility related to the procedure, and (iv) the status of the plurality of tasks required to advance the patient through the procedure.

14. The method of claim 1, further comprising automatically updating the live dataset with correction of the deficiency.

15. The method of claim 1, further comprising the healthcare worker manually updating the live dataset with correction of the deficiency.

16. The method of claim 1, further comprising:
calculating a safety score for the procedure for the patient based on the live dataset; and
calculating an updated safety score for the procedure for the patient based on correcting the deficiency and an update to the live dataset.

17. A system for presenting live status of a first procedure for a first patient to personnel in a healthcare facility, comprising:
a processor accessing a live dataset including (i) a status of the first patient, (ii) a status of a plurality of healthcare assets related to the first procedure, (iii) a status of a plurality of infrastructure elements of the healthcare facility related to the first procedure, and (iv) a status of a plurality of tasks required to advance the first patient through a workflow of the first procedure;
a visual display, informationally coupled with the processor, displaying a plurality of elements from the live dataset including at least part of (i) the status of the first patient, (ii) the status of the plurality of healthcare assets related to the first procedure, (iii) the status of the plurality of infrastructure elements of the healthcare facility related to the first procedure, and (iv) the status of the plurality of tasks required to advance the first patient through the workflow of the first procedure;
a communication module, informationally coupled with the processor, notifying a healthcare worker associated with a first task from the plurality of tasks of a deficiency related to the first task identified in the live dataset;
correcting, by the healthcare worker, the deficiency; and
the visual display displaying a plurality of elements from the live dataset including the correcting the deficiency.

18. The system of claim 17, further comprising presenting live status of a second procedure for a second patient, wherein the live dataset further comprises (i) a status of the second patient, (ii) a status of a plurality of healthcare assets related to the second procedure, (iii) a status of a plurality of infrastructure elements of the healthcare facility related to the second procedure, and (iv) a status of a plurality of tasks required to advance the second patient through a workflow of the second procedure, and wherein live status of the second patient is derived from the live dataset.

19. The system of claim 18, wherein the first procedure for the first patient is scheduled to occur before the second procedure for the second patient, and wherein the first procedure and the second procedure require at least one common healthcare facility asset.

20. A method for advancing a patient through a workflow of a procedure in a healthcare facility, comprising:
accessing a live dataset including (i) a status of the patient, (ii) a status of a plurality of healthcare assets related to the procedure, (iii) a status of a plurality of infrastructure elements of the healthcare facility related to the procedure, and (iv) a status of a plurality of tasks required to advance the patient through the workflow of the procedure;
simultaneously notifying a first healthcare worker associated with a first task in a first step of the workflow of a deficiency related to the first task, and a second healthcare worker of a second task in a subsequent second step in the workflow of the procedure of a deficiency related to the second task, and
the first and second health workers correcting the deficiencies in the first and second tasks, respectively, wherein the patient is cleared to progress from the first step of the workflow, through the second step of the workflow, to a subsequent third step in the workflow.
